(19) United States
(12) Patent Application Publication
(10) Pub. No.: US 2002/0179095 A1
(43) Pub. Date: Dec. 5, 2002

(54) Method for diagnosis of chronic headache

(76) Inventor: Irina G. Almazov, Kfar Saba (IL)

Correspondence Address: DR. MARK FRIEDMAN LTD, c/o BILL POLKINGHORN - DISCOVERY DISPATCH, 9003 FLORIN WAY, UPPER MARLBORO, MD, 20772, US

(21) Appl. No.: 09/865,681

(22) Filed: May 29, 2001

(51) Int. Cl. A61B 19/00

(52) U.S. Cl. 128/898

(57) ABSTRACT

A method for determining a cause of chronic headache is disclosed. The method includes: (a) determining whether the headache of a person is postural, (b) determining whether the person has meningeal irritation, and (c) determining that the headache is caused by intracranial hypotension if the headache is postural and the person has meningeal irritation.

### FIELD AND BACKGROUND OF THE INVENTION

The present invention relates to clinical medical diagnosis and, more particularly, to a method for determining a cause of chronic headache.

Headache is felt to be the most common human malady and the most prevalent neurologic symptom associated with any disease. Without question, the most frequent of all the painful states that afflict humans, it rivals backache as the most common reason for medical consultation. It accounts for almost 20 million outpatient visits a year in the United States. and is a leading cause for the use of over-the-counter medications.

Headache is a major social and economic burden for society. In children, headache is an extremely common complaint. Depending on the etiology, frequency and intensity of the headache, headaches can have a major effect on a child's school attendance and academic performance, as well as the child's memory, personality and interpersonal relationships.

Headache presents a diagnostic challenge to the physician, and can be a very difficult problem to evaluate and manage. Only one third of patients who seek medical assistance for this symptom claim they are satisfied with the care received. Although headache is usually a benign, primary medical disorder, it can be the symptom of a serious and life threatening disease. The physician must diagnose the type of headache, systematically identify the cause of the headaches, and identify those patients who require aggressive evaluation. Symptomatic relief must be provided and a long-term plan for management, including prevention, must be formulated.

With migraine and tension-type headaches being the most common causes of chronic or recurrent headaches, and brain tumor being the most feared, there exists a complex differential diagnosis of chronic headache. The International Headache Society published, in 1988, a comprehensive classification system and formal criteria for the diagnosis of headache disorders (Headache Classification Committee of the International Headache Society (1988) Cephalalgia 8 (suppl. 7): 1-96) that have achieved common acceptance. Tension-type headaches are the overall most common variety of headache. There is a lifetime prevalence of tension-type headaches of 69 percent in men and 88 percent in women. Whereas almost 60 percent of the population may have episodic tension-type headaches in any one year, about three percent of the population suffers from the chronic type, with daily headache. Tension-type headaches, while occurring in all age groups, are said to be comparatively uncommon in childhood, particularly before puberty, but the prevalence of tension-type headaches increases significantly in adolescent patients (Aicardi J (1992) Diseases of the Nervous System in Childhood London: MacKeith Press, pp. 1021-2; and Behrman R E et al (2000) Nelson Textbook of Pediatrics, 16^{th} edition, Philadelphia: W.B. Saunders, pp. 1834-5). The onset of tension-type headaches during adolescence or young adulthood is most common in fact. Overall migraine headaches are held to be the most frequent and important cause of headaches in the pediatric population.

Headache is often divided into acute and chronic/recurrent/persistent types and into primary (or benign) and secondary (or symptomatic) forms. Overall, there is no known or identified cause for the majority of patients with chronic, recurrent daily headaches who don't have an underlying neurologic problem.

Textbooks of pediatrics and neurology identify the major secondary causes of headache in children as increased intracranial pressure resulting from brain tumors, hydrocephalus, meningitis and encephalitis, cerebral abscess, subdural hematoma, chronic lead poisoning, and pseudotumor cerebri; as well as those that may not be associated with increased intracranial pressure including vascular malformations, berry aneurysm, connective tissue diseases, arterial hypertension, acute subarachnoid hemorrhage and stroke (Behrman R E et al (2000) Nelson Textbook of Pediatrics, 16^{th} edition, Philadelphia: W.B. Saunders, pp. 1832-4; Aicardi J. (1992) Diseases of the Nervous System in Childhood London: MacKeith Press, p. 1022). Refractive errors, strabismus, sinusitis and malocclusion of the teeth are described as much less common causes of significant headaches in children.

Decreased intracranial pressure, or intracranial hypotension, has been identified and recognized as a cause of acute headache syndromes and has been associated with specific causes. A major pediatric handbook, The Harriet Lane Handbook, for example, lists decreased intracranial pressure among the differential diagnosis of symptomatic headache. This handbook states that symptomatic headache is due to serious intracranial or systemic disease and benign headache is not. It lists the causes of decreased intracranial pressure as: after ventriculoperitoneal shunt, lumbar puncture, or cerebrospinal leak from basilar skull fracture.

The International Headache Society classification lists headaches caused by low cerebrospinal fluid pressure as occurring only in two situations: post-lumbar puncture headache, and cerebrospinal fluid fistula headache. The latter results in occult cerebrospinal fluid leakage with effusion, and consequently, decreased cerebrospinal fluid volume with low cerebrospinal fluid pressure, i.e., intracranial hypotension.

Intracranial hypotension has been divided into two categories: spontaneous, with no evidence of a cerebrospinal fluid leak or systemic illness, and symptomatic, which may be associated with a cerebrospinal fluid leak. Symptomatic causes, in addition to those listed above, include head or back trauma, complications of cranio-spinal surgery, and certain systemic medical illnesses such as “severe dehydration,” diabetic coma, meningoencephalitis, uremia, “severe systemic infection,” and infusion of hypertonic solutions. (Silberstein S, Headache 1992, 32: 84-94).

The prior art has not recognized or identified intracranial hypotension as a cause or, more particularly, the major (non-migraine) cause of chronic headache, particularly in children and adolescents, nor has it provided a method for identifying intracranial hypotension as the cause of chronic headaches in an individual child or adolescent. Studied publications have revealed no data concerning the effect of intracranial hypotension on the development of chronic headache in children. There is thus a recognized need for, and it would be highly advantageous to have, a method for the clinical identification of the cause of chronic headache in individuals, particularly children, devoid of the above limitation. Such a method would allow proper prediction of the natural history of the headaches and importantly would guide the selection of appropriate strategies for symptom management and prevention.

Post-lumbar puncture headache, and the headaches of cerebrospinal fistulae and spontaneous intracranial hypotension, have been recognized as orthostatic or postural headaches. According to the International Headache Society classification, the diagnostic criteria for post-lumbar puncture headache and the headache of cerebrospinal fistulae includes the following: “The headache occurs or worsens less than 15 minutes after assuming the upright position and disappears or improves less than 30 minutes after resuming the recumbent position” (Headache Classification Committee of the International Headache Society (1988) Cephalalgia 8 (suppl. 7): p 52).

Pathologic changes have been identified in the cerebral membranes in cases of acute headache due to spontaneous intracranial hypotension. Lymphocytic pleocytosis and/or elevated cerebrospinal fluid protein levels have been described in cerebrospinal fluid examination of such affected individuals (Arjona A et al (1998) Headache 38: 142-3; Mokri B et al (1995) Neurology 45: 1801-7; Pannulosc-Reich J B et al (1993) Neurologia 43: 919-26; Kinoshita F et al (1997) No Shinkei Geka 25: 437-42). Magnetic resonance imaging (MRI) of the brain has revealed the presence of diffuse pachymeningeal gadolinium enhancement (Marto-Norena E et al (1998) Rev Neurol 158: 589-91; Rabin B M et al (1998) Am J Neuroradiol 19: 1034-9; O'Carolli C P et al (1999) Cephalalgia 19: 80-7; Fedder S L (1999) Spine 24: 463-4; Bamg O Y et al (1998) Eur Neural 39: 49-56; Benamot N (1998) Clini Nucl Med 23: 150-1; Asci N et al (1998) Acta Neurochiz Wien 140: 47-50). Meningeal biopsy has confirmed the presence of pachymeningitis in spontaneous intracranial hypotension (Mokri B et al (1995) Neurology 45: 1801-7; Niwa K et al (1997) No To Shinkei 49: 541-6; Beck C E et al (1998) J Neurosurg 89: 470-3). Mokri et al. (Mokri B et al (1995) Neurology 45: 1801-7) described the meningeal biopsy in spontaneous intracranial hypotension as demonstrating non-specific granulation tissue with mild inflammatory changes. The authors speculate “that the inflammatory changes of the dural border cell layer correspond to the zone of pachymeningeal gadolinium enhancement of the brain MRI. The granulation tissue of the dural border cell layer and subdural fluid accumulation may represent secondary reactive phenomena.” Case reports of acute headaches in spontaneous intracranial hypotension have included descriptions of cervical pain (Moreno M Y (1996) Neurologia 11: 236-8) and nuchal rigidity (Yhara M et al (1998) Rinsho Shin Kedaku 38: 834-42; Marto-Norena E et al (1998) Rev Neurol 158: 589-91) as well as disturbances of the abducens and acoustic nerves (Arjona A et al (1998) Headache 38: 142-3; Yhara M et al (1998) Rinsho Shin Kedaku 38: 834-42; Dumont D (1998) Rev Rheum Eng Ed 65: 352-4; Arjon A et al (1997) An Otorrinolaringal Ibero Am 24: 505-9; Berlit P et al (1994) Neurology 44: 1552). No similar findings have been identified previously in cases of chronic headache in children and adolescents.

Intracranial pressure measurements by lumbar puncture as the primary indicator of intracranial hypotension have been inconsistent. One review (Silberstein S, Headache 1992, 32: 84-94) states that cerebrospinal fluid pressure measurements by lumbar puncture in cases of intracranial hypotension are found to be within the range of 0-30 mm H₂O in the lateral decubitus position, while another paper (Arjona A et al (1998) Headache 38: 142-3) considers that cerebrospinal fluid pressure measurement by lumbar puncture is not an absolute indicator of intracranial hypotension as it depends on horizontal or vertical positioning of the patient during the 1-2 hours immediately prior to the puncture.

Several remedies have been suggested to raise intracranial pressure, including continuous epidural saline infusion (Kawasaki S et al (1999) No To Shinkei 51: 711-5; Ramadan N M (1996) Curr Opin Neurol 27: 1027-33), and vasoconstrictor administration, particularly caffeine (Tkhurana R K (1996) Semin Neurol 16: 5-10; Ramadan N M (1996) Curr Opin Neurol 27: 1027-33; Arjona A (1998) Rev Neurol 160: 1027-33). Further recommendations include corticosteroids or mineralocorticoids (Ramadan N M (1996) Curr Opin Neurol 27: 1027-33) and theophylline (Raskin N H (1990) Headache 30: 197). Non-pharmacological conservative treatments include maintaining an affected patient horizontally in bed for several days with abundant liquid consumption (Silberstein S, Headache 1992, 32: 84-94; Lay C L et al in Goadsby, P J et al Blue Book of Practical Neurology: Headache (1997) Boston: Butterworth-Heinemann, pp. 355-67; Jedanak C P et al (1986) Re Neurol Paris 1142: 937-8). This latter method is used frequently in hospital practice to “cure” post-lumbar puncture headache, in particular. Treatment of intracranial hypotension has not been previously suggested as a remedy for chronic headache in children, nor has the response to such maneuvers been proposed previously as a diagnostic adjunct to identifying the cause of chronic headaches in children.

There is thus a recognized need for, and it would be highly advantageous to have, a method for the clinical identification of the most common cause of chronic, non-migraine, headache in children.

### SUMMARY OF THE INVENTION

According to one aspect of the present invention there is provided a method for determining a cause of chronic headache including: (a) determining whether the headache of a person is postural; (b) determining whether the person has meningeal irritation; and, (c) determining that the headache is caused by intracranial hypotension if the headache is postural and the person has meningeal irritation.

According to another aspect of the present invention there is provided a method for determining a cause of chronic headache including: (a) determining whether the headache of a person is postural; (b) determining whether the person has meningeal irritation; (c) determining whether the person has at least one sign of acoustic nerve involvement; and, (d) determining that the headache is caused by intracranial hypotension if the headache is postural, if the person has meningeal irritation, and if the person has at least one sign of acoustic nerve involvement.

According to yet another aspect of the present invention there is provided a method for determining a cause of chronic headache including: (a) determining whether the headache of a person is postural; (b) determining whether the person has meningeal irritation; (c) determining whether the headache is relieved after the person is treated with a specific treatment regimen; and, (d) determining that the headache is caused by intracranial hypotension if the headache is postural, the person has meningeal irritation, and the headache is relieved after the person is treated with the specific treatment regimen.

According to further features in preferred embodiments of the invention described below, determining whether the person has meningeal irritation includes performing at least one physical examination maneuver for ascertainment of the presence of at least one meningeal sign.

According to still further features in the described preferred embodiments, the at least one meningeal sign is selected from the group consisting of nuchal rigidity, pain with forward flexion of the head, Kernig sign, Brudzinski upper sign, Brudzinski middle sign, Brudzinski lower sign, tripod sign, Guillain sign, and facial symptoms.

According to still further features in the described preferred embodiments, determining whether the headache is postural includes determining whether an intensity of the headache increases when the person moves from a recumbent position to an upright position.

According to still further features in the described preferred embodiments, determining whether the headache is postural includes further determining whether the intensity increases within 15 minutes of moving to the upright position.

According to still further features in the described preferred embodiments, determining whether the headache is postural includes determining whether the intensity of the headache decreases when the child moves from an upright position to a recumbent position.

According to still further features in the described preferred embodiments, determining whether the headache is postural further includes determining whether the intensity decreases within 30 minutes of moving to the recumbent position.

According to still further features in the described preferred embodiments, the person is a child.

According to still further features in the described preferred embodiments, the person is an adult.

According to still further features in the described preferred embodiments, the at least one sign of acoustic nerve involvement is selected from the group consisting of dizziness, phonophobia, partial unilateral hearing reduction, and disturbance of balance.

According to still further features in the described preferred embodiments, the specific treatment regimen includes the person remaining in a recumbent position for a prolonged period of time.

According to still further features in the described preferred embodiments, the prolonged period of time is at least two days.

According to still further features in the described preferred embodiments, the specific treatment regimen includes the person consuming an abundant amount of a fluid.

According to still further features in the described preferred embodiments, the abundant amount of the fluid is at least 50 milliliters of the fluid per kilogram of body weight of the person per day.

The present invention successfully addresses the shortcomings of the presently known configurations by providing a method for determining a cause of chronic headache in an individual. The method of the present invention provides a method for determining that a chronic benign headache is caused by intracranial hypotension. The present invention further provides a method for the clinical identification of the most common cause of chronic, non-migraine, headache in children. The method of the present invention allows proper prediction of the natural history of the headaches and importantly guides the selection of appropriate strategies for symptom management and prevention.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic diagram illustrating a classification of headaches;

FIG. 2 schematically illustrates the Kernig and Brudzinski lower signs;

FIG. 3 is a graph showing the frequencies of different meningeal signs in a study population of children with chronic, non-migraine, headache; and,

FIG. 4 is a table showing the frequencies of different meningeal signs in a study population of children with chronic, non-migraine, headache.

### DESCRIPTION OF THE PREFERRED EMBODIMENTS

The present invention is of a method which can be used to diagnose a cause of chronic headaches, including chronic headaches in children and adolescents. Specifically, the present invention can be used as a method to identify individuals, including children and adolescents, with a chronic headache due to intracranial hypotension.

For purposes of this specification and accompanying claims, the term, “headache” is defined as any ache, pain or discomfort located in the region of the cranial vault, above the orbitomeatal line.

For purposes of this specification and accompanying claims, the terms, “decreased intracranial pressure”, “intracranial hypotension”, “low cerebrospinal fluid pressure” and “decreased cerebrospinal fluid pressure” are used interchangeably to mean a level of intracranial cerebrospinal fluid pressure below normal.

For purposes of this specification and accompanying claims, the terms, “child” and “children” are used to refer to an individual under 18 years of age, and includes the term “adolescent” which refers to an individual from 12-18 years of age. For purposes of this specification and accompanying claims, the term, “pediatric” is used to refer to health care delivered to individuals under the age of 18. For purposes of this specification and accompanying claims, the term “patient” is used to refer to an individual who is awaiting or under the care of a health care professional and includes individuals who are suffering from some symptom or disorder even if not under the care of a health care professional.

For purposes of this specification and accompanying claims, the term “chronic headache” encompasses both persistent and recurrent headaches and refers to a headache or recurrent headaches that occur over a prolonged period of time, generally at least several weeks to months or even years.

The principles and operation of a method which can be used to diagnose a cause of chronic headaches in individuals, including but not limited to, children and adolescents, according to the present invention may be better understood with reference to the drawings and accompanying descriptions.

Before explaining at least one embodiment of the invention in detail, it is to be understood that the invention is not limited in its application to the particular details and arrangement set forth in the following description or illustrated in the drawings. On the contrary, the invention is capable of other embodiments or of being practiced or carried out in various ways, and it is intended to cover all alternatives, modifications and equivalents as may be included within the scope of the invention as defined by the appended claims. Also, it is to be understood that the phraseology and terminology employed herein is for the purpose of description and should not be regarded as limiting.

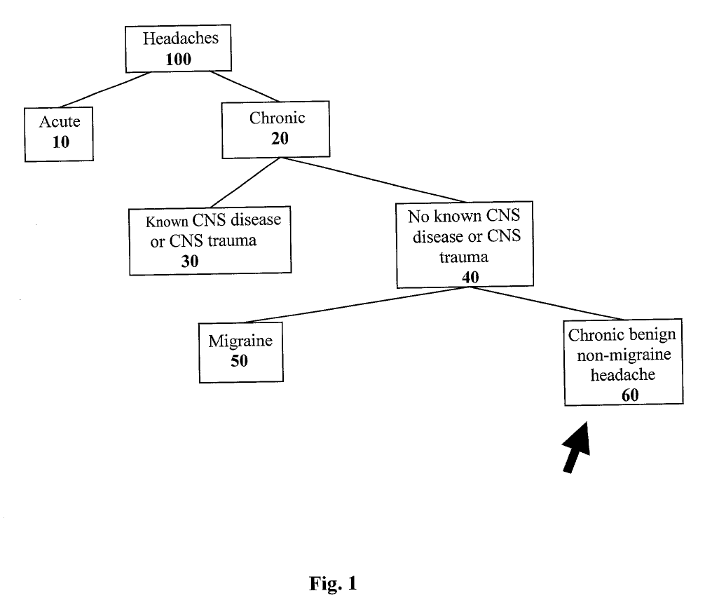

Referring now to the drawings, FIG. 1 schematically illustrates a classification of headaches. Headaches 100 are first divided into acute headaches 10 and chronic headaches 20. Chronic headaches 20 are then divided into headaches 30 in patients with an identified organic central nervous system (CNS) disease or CNS trauma and headaches 40 in patients with no identified organic central nervous system (CNS) disease or CNS trauma. Chronic headaches in patients with no identified organic central nervous system (CNS) disease or CNS trauma 40 are further sub-divided into migraine headaches 50 and into chronic non-migraine headaches in patients with no identified organic central nervous system (CNS) disease or CNS trauma 60.

The method of the present invention is used to diagnose a cause of chronic non-migraine headaches with no identified organic central nervous system (CNS) disease or CNS trauma, that is, headaches of this latter group, 60, (as indicated by the arrow in FIG. 1) which for the purposes of this specification and the accompanying claims, are referred to as chronic benign non-migraine headaches. The method of the present invention permits the identification and diagnosis of intracranial hypotension as a cause for chronic headache in, particularly, but not limited to, children and adolescents. The method of the present invention uses a clinical assessment of a person affected with a chronic headache. The clinical assessment includes a detailed headache history (including ascertainment of chronicity, frequency, time of occurrence, exacerbating and ameliorating factors, preceding and accompanying symptoms, pain location, and pain quality), a physical examination (including arterial blood pressure determination), and neurological examination.

According to the method of the present invention, a person suffering from chronic benign non-migraine headaches is evaluated to determine whether the person's headache is postural in nature. The evaluation to determine whether the person's headache is postural is based on the medical history obtained as part of the clinical assessment. Alternatively, the determination that the person's headache is postural may be based on physical examination maneuvers involving evaluation of the headache presence and intensity in different positions, such as recumbent and upright. Evaluation of headache intensity utilizes, for example, such reporting and rating scales as are known in the art, including self-rating scales and those which use graphical and visual representations of pain intensity, as non-limiting examples. A postural headache, also known as an orthostatic headache, is one which occurs or worsens immediately or soon (preferably within 15 minutes) after arising and assuming an upright posture. A postural (orthostatic) headache typically disappears or preferably improves soon (preferably less than 30 minutes) after resuming a recumbent position.

In the usual pediatric and neurologic practice, a child with chronic headaches is subjected to a complete physical and neurological examination. The routine and customary examination of a child with chronic headaches does not however include an assessment for the presence of meningismus. In accordance with the method of the present invention, a person suffering from chronic benign non-migraine headaches is evaluated to determine whether the person has evidence of meningeal irritation. The assessment for the presence of meningeal irritation includes at least one evaluation aimed at elicitation of at least one meningeal sign. The at least one meningeal sign includes: (1) presence of nuchal rigidity, (2) Kernig sign, (3) Brudzinski signs, (a) upper, (b) middle, (c) lower, (4) Guillain sign, (5) tripod sign, (6) elicitation of pain on forward flexion of the head, and (7) pain elicited by pressure on at least one trigeminal point, the trigeminal points including the projection of the supraorbital foramina, and the infraorbital foramina as well as Bechterev sign, a facial grimace expressing pain with percussion along the zygoma of the corresponding hemi-face, referred to for the purposes of the specification and the accompanying claims as “facial symptoms”.

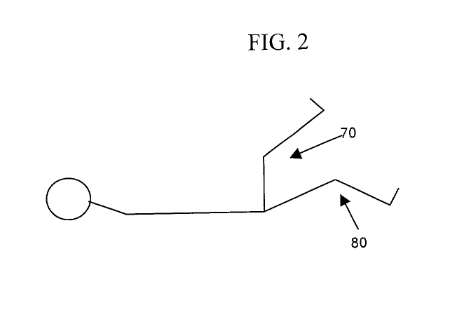

The test for Kernig sign is accomplished by having the patient lie supine, flexing the thigh 90 degrees at the hip joint so that it is at a right angle to the trunk (while simultaneously maintaining the knee flexed), and then straightening and extending the leg at the knee joint, with the hip remaining flexed. If the leg cannot be completely extended due to posterior thigh muscle pain and reflex spasm of the hamstrings, as schematically illustrated as 70 in FIG. 2, this is Kernig sign.

The Brudzinski sign is a group of symptoms of meningeal irritation. The Brudzinski upper sign is elicited in a patient lying supine by passively forward flexing the head toward the chest. The Brudzinski upper sign is the flexion of the lower limbs at the hip and knee that occurs in response to the neck flexion. The Brudzinski middle (or pubic) sign is elicited by pressure on the pubic symphysis, which causes flexion of the lower extremities at the hips and knees and adduction of the hips. The Brudzinski lower sign is reflex flexion of the lower extremity following passive flexion of the contralateral lower extremity, as schematically illustrated as 80 in FIG. 2.

The Guillain sign is leg flexion that occurs instantly upon pinching of the contralateral quadriceps tempri muscle. When patients with meningeal irritation are unable to sit straight with extended, straightened lower extremities and rest supported on their hands and upper extremities extended behind their trunk, this is known as the tripod sign. The tripod sign may be both passively observed or actively elicited on examination. Pain accompanying passive or active forward flexion of the neck may be elicited at the occiput, in the neck, epigastrium, back or chest. Nuchal rigidity is defined as stiffness and spasm of the muscles of the occiput and neck. Nuchal rigidity is assessed by placing the examiner's hand under the patient's head and gently attempting to flex the neck toward the chest. Undue resistance defines nuchal rigidity and implies diffuse irritation of the cervical nerve roots from meningeal inflammation.

According to the method of the present invention a person with a chronic, benign, non-migraine headache is diagnosed as having intracranial hypotension as the cause of the headache if the person has the combination of a postural headache and evidence of meningeal irritation. According to a preferred embodiment of the present invention, evidence of meningeal irritation includes the elicitation of at least one meningeal sign.

According to the method of the present invention, additional support for the diagnosis of intracranial hypotension is achieved with elicitation of symptoms of involvement of the acoustic nerve (eighth cranial nerve), including dizziness, phonophobia, partial unilateral hearing reduction and mild truncal ataxia. According to the method of the present invention, further support for the diagnosis of intracranial hypotension is achieved if there is a reduction or relief of headache symptoms and reduction or complete disappearance of meningeal signs and symptoms with a treatment regimen consisting of remaining in a horizontal, recumbent position for several days and abundant fluid consumption.

Additional objects, advantages, and novel features of the present invention will become apparent to one ordinarily skilled in the art upon examination of the following example, which is not intended to be limiting. Additionally, each of the various embodiments and aspects of the present invention as delineated hereinabove and as claimed in the claims section below finds experimental support in the following example.

### EXAMPLE

Reference is now made to the following example, which together with the above descriptions, illustrates the invention in a non-limiting fashion.

A clinical study was undertaken with over one thousand children with a chief complaint of headache evaluated as outpatients by a pediatric neurologist to whom they were referred by their primary care physician. Patients with known organic CNS disease and brain trauma were excluded from this study. The patients ranged in age from 7 to 17 years old, were all attending school, and were of equal sex distribution. The patients were broadly representative of all the major ethnic groups seen in Israel, including Ashkenazi and Sephardic Jews, Arabs, Ethiopians, ethnic Russians and others.

A twenty-three item questionnaire was administered to elicit the headache history. All patients had been previously evaluated by their pediatrician or family physician. The physical examinations and states were normal and their arterial pressure determinations were within or slightly below age appropriate normal ranges. Routine laboratory evaluations of blood and urine were normal and sinus x-rays excluded sinusitis as a cause of the headaches. A full neurologic examination was undertaken with special attention to testing for signs of meningeal irritation as delineated hereinabove.

In sixty percent of the patients evaluated, the character of the headache and clinical description corresponded to different types of migraine. In none of these patients, were any meningeal signs present.

The remainder of the study concentrated on the remaining 40% of patients who had headaches characterized as non-migraine in nature, hereinafter referred to as the study population. These patients had been complaining about headaches for several weeks, months, and even for three years. The headaches in the study population were described as continuous and daily, or recurrent periodically through the week, but during any given day, periods of worsening and relief were marked. The majority of the patients (92%) had no prior history of headaches. In the 8% that had previously suffered from headaches, they previously were short-lived and self-limited, and they had family members who also had a tendency to headache.

In the study population, the headache of all patients occurred in the morning, immediately or soon after arising; i.e., the character of the headache was orthostatic, that is, postural. All patients noted headache disappearance on reclining to a recumbent position. As a rule, the headache localized in the forehead and frontal region from both sides, but occasionally it spread over the occiput. The patient in the study population typically described it as “pressing” or “pulsing”.

In 90% of patients in the study population, the headache was accompanied by dizziness that caused an abrupt and unpleasant “dizziness inside the head”. Only 2% of the patients had episodes of systemic, or peripheral, dizziness, that is, a sensation of true vertigo. Seven percent of the children occasionally vomited but no clear connection between the vomiting and the headache was detected. Seventy-three percent of patients experienced phonophobia accompanying worsening of the headache. Eighty-three percent of subjects in the study population complained of periodic abdominal pains.

On questioning, 10% of the children and their parents mentioned some “acute” illness, more often of viral origin, which might have been connected to the initial period of headache. The headaches neither were exacerbated nor ameliorated during or after physical exertion, such as sports lessons. The general state of the patients in the study population was satisfactory, and all of them were able to attend classes in spite of daily headache. There were, however, days when the headaches worsened and individual children had to leave their classes.

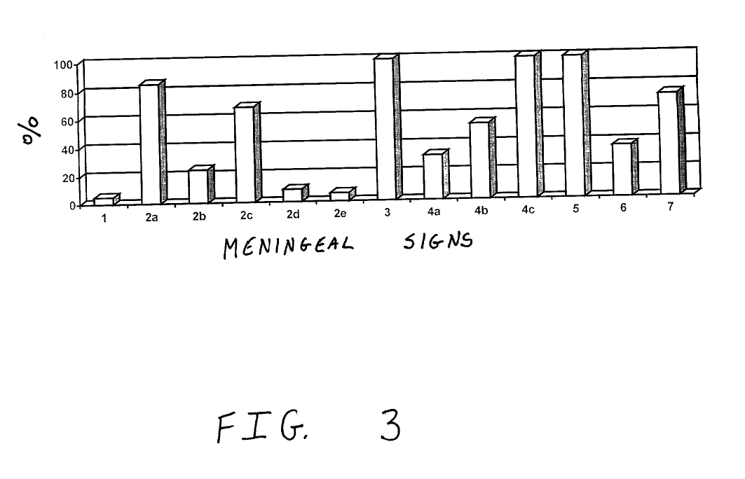

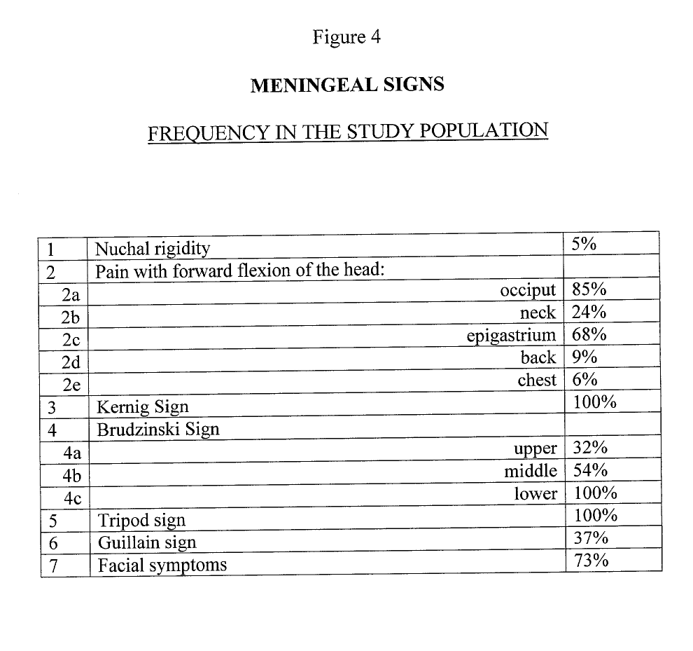

The neurological examinations revealed that all patients in the study population had at least one meningeal sign as described hereinabove. The frequencies of the various meningeal signs in the study population are shown in FIG. 3 and in tabular form in FIG. 4. Along the x-axis of FIG. 3 are the various meningeal signs tested, numbered as indicated in FIG. 4.

While only 5% of the patients showed any rigidity of the occipital muscles, all patients complained of pain in the head or neck on forward flexion of the head. The Kernig sign and Brudzinski lower sign were detected in all patients. The tripod sign was also universally observed. Other signs were observed variably in individual patients. In 60% of the patients describing dizziness, an average 22% unilateral hearing loss was revealed on neurological examination. All patients demonstrated some degree of truncal ataxia with misbalance and rocking on Romberg test and “tandem” walking. No case of palsy of the abducens (sixth cranial) nerve was detected. Ophthalmoscopy was performed on all patients; the fundus was unchanged in all and spontaneous pulsation of retinal vessels was registered in all. In 6% of the subjects of the study population a CT scan of the brain was performed and revealed no pathology.

The results of the clinical assessment with assessment of the medical history and evaluation of meningeal signs allowed for the diagnosis of all patients in the study population as having intracranial hypotension. On this basis, all of the patients in this study population were treated with a treatment regimen consisting of remaining in a horizontal, recumbent position for several days and abundant fluid consumption (50 ml/kg body weight per day). In all patients who followed these recommendations (77%) there was a significant diminution of headache intensity and dizziness in 2-3 days and complete resolution within 7-10 days. The patients returned to their normal activity and 86% suffered no further recurrence of symptoms. Re-examination of patients whose headaches resolved revealed disappearance of meningeal signs within 7-10 days after the initiation of the treatment regimen described hereinabove. In 8% of the study population, patients had additional features of migraine type headaches. In this subgroup of patients, after treatment and termination of the postural headaches and disappearance of the meningeal signs, migraine type headaches persisted.

This study suggests that intracranial hypotension is a frequent cause of headaches in children and adolescents. On the basis of this data it is concluded that intracranial hypotension is the second most frequent cause after migraine of chronic headaches in children.

Analysis of the headache characteristics of the study population suggests that the headaches in the study population meet published criteria for chronic tension headache (also known as chronic daily headache). (Headache Classification Committee of the International Headache Society (1988) Cephalalgia 8 (suppl. 7): 1-96; Silberstein S D et al (1994) Headache 34: 1-7; Silberstein S D (1993) Neurology 43: 1644-49; Gladstein J and Holden E W (1996) Headache 36: 349-51; Saper J (1990) Neurol Clin 8: 891-901). The headache duration, pressing or pulsing character, bilateral localization, absence of headache worsening with physical activity, the presence of either only phonophobia or photophobia or their complete absence, and the absence of nausea and vomiting are the criteria shared by the headaches of the study population and the defining criteria of chronic tension headache. The diagnostic criteria in the prior art do not refer to either the postural nature of the headache or evidence of meningeal irritation in chronic type headache. Thus specifically envisioned as being within the scope of the present invention are alternate configurations in which the method of the present invention is utilized in patients older than the pediatric age group, that is alternate configurations in which the method of the present invention is used to determine a cause of a headache in an individual over the age of 18, and in particular an individual of any age with a chronic tension type headache.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method for determining a cause of chronic headache comprising:
(a) determining whether the headache of a person is postural;
(b) determining whether said person has meningeal irritation; and,
(c) determining that the headache is caused by intracranial hypotension if the headache is postural and said person has meningeal irritation.

2. The method of claim 1, where said determining whether said person has meningeal irritation includes performing at least one physical examination maneuver for ascertainment of presence of at least one meningeal sign.

3. The method of claim 2, where said at least one meningeal sign is selected from the group consisting of nuchal rigidity, pain with forward flexion of the head, Kernig sign, Brudzinski upper sign, Brudzinski middle sign, Brudzinski lower sign, tripod sign, Guillain sign, and facial symptoms.

4. The method of claim 1, where said determining whether the headache is postural includes determining whether an intensity of the headache increases when said person moves from a recumbent position to an upright position.

5. The method of claim 4, where said determining whether the headache is postural includes determining whether said intensity increases within 15 minutes of moving to said upright position.

6. The method of claim 1, where said determining whether the headache is postural includes determining whether an intensity of the headache decreases when the child moves from an upright position to a recumbent position.

7. The method of claim 6, where said determining whether the headache is postural further includes determining whether said intensity decreases within 30 minutes of moving to said recumbent position.

8. The method of claim 1, where said person is a child.

9. The method of claim 1, where said person is an adult.

10. A method for determining a cause of chronic headache comprising:
(a) determining whether the headache of a person is postural;
(b) determining whether said person has meningeal irritation;
(c) determining whether said person has at least one sign of acoustic nerve involvement; and,
(d) determining that the headache is caused by intracranial hypotension if the headache is postural, if said person has meningeal irritation, and if said person has said at least one sign of acoustic nerve involvement.

11. The method of claim 10, where said at least one sign of acoustic nerve involvement is selected from the group consisting of dizziness, phonophobia, partial unilateral hearing reduction, and disturbance of balance.

12. A method for determining a cause of chronic headache comprising:
(a) determining whether the headache of a person is postural;
(b) determining whether said person has meningeal irritation;
(c) determining whether the headache is relieved after said person is treated with a specific treatment regimen; and,
(d) determining that the headache is caused by intracranial hypotension if the headache is postural, if said person has meningeal irritation, and if the headache is relieved after said person is treated with said specific treatment regimen.

13. The method of claim 12, where said specific treatment regimen includes said person remaining in a recumbent position for a prolonged period of time.

14. The method of claim 13, where said prolonged period of time is at least two days.

15. The method of claim 12, where said specific treatment regimen includes said person consuming an abundant amount of a fluid.

16. The method of claim 15, where said abundant amount of said fluid is at least 50 milliliters of said fluid per kilogram of body weight of said person per day.
